(19) United States
(12) Patent Application Publication
(10) Pub. No.: US 2026/0209678 A1
(43) Pub. Date: Jul. 23, 2026

(54) FERMENTATION DEVICE

(71) Applicant: Chandar Kamalanathan, Austin, TX (US)

(72) Inventor: Chandar Kamalanathan, Austin, TX (US)

(21) Appl. No.: 19/414,518

(22) Filed: Dec. 10, 2025

Related U.S. Application Data
(60) Provisional application No. 63/746,575, filed on Jan. 17, 2025.

(51) Int. Cl. C12M 1/02; A21C 13/00; C12M 1/00; C12M 1/36; C12M 3/00

(52) U.S. Cl. CPC C12M 41/22; A21C 13/00; C12M 23/14; C12M 23/38; C12M 23/48; C12M 41/48

(57) ABSTRACT

A fermentation device comprises a fermentation container housed within a multilayer insulation bag. The fermentation container includes a basin and a lid that define an interior volume for holding fermentable material. The insulation bag includes five layers: a heat-dampening lining fabric, an integrated heated mat, an insulation film, a cotton insulation layer, and a waterproof outer layer. A controller operatively connected to the heated mat includes a user interface and a timer for regulating fermentation parameters. A temperature sensor having a probe extends into the interior volume and continuously communicates internal temperature data to the controller. Based on the received temperature data, the controller automatically adjusts power to the heated mat to maintain a selected fermentation temperature. The multilayer insulation structure enhances thermal retention and stability, enabling precise and consistent temperature control during fermentation in a compact device suitable for small-scale or residential environments.

### FIELD OF THE DISCLOSURE

The present disclosure relates generally to fermentation systems and devices. More particularly, the disclosure pertains to temperature-controlled fermentation devices incorporating integrated heating elements, multilayer insulation structures, and sensor-based thermal regulation for maintaining stable fermentation conditions, including conditions suitable for fermenting liquid dough.

### BACKGROUND OF THE RELATED ART

Fermentation is a biological process in which microorganisms such as yeast and bacteria convert carbohydrates into alcohols, acids, and other byproducts under anaerobic conditions. This process underlies numerous culinary and industrial applications, including the preparation of bread, beer, wine, yogurt, kimchi, sauerkraut, and various fermented foods. In particular, the fermentation of liquid dough is used to produce baked goods such as pancakes, waffles, crêpes, and sourdough products, and requires controlled temperature conditions to promote consistent yeast activity and the development of desired flavor and textural characteristics.

Liquid dough fermentation in commercial settings is typically performed using large-scale fermentation equipment equipped with precision temperature regulation, humidity control, automated mixing systems, and digital monitoring interfaces. While such equipment provides consistency and repeatability in professional environments, its large physical dimensions, high costs, complex installation requirements, and operational sophistication render it unsuitable for household use.

Household fermentation practices, by contrast, generally rely on improvised techniques that lack precision and reproducibility. Common methods include covering bowls with cloths or plastic wraps and placing them near heat sources such as ovens, radiators, or sunny windowsills, or immersing containers in warm water baths that require periodic temperature monitoring. Some users attempt to warm dough by leaving oven lights on or using low oven settings. These approaches are labor-intensive, sensitive to ambient environmental variations, and prone to producing inconsistent fermentation outcomes.

Environmental factors such as room temperature, humidity, airflow, and heat-source stability are difficult to control in domestic settings. Variations in these factors often result in uneven fermentation rates, undesirable off-flavors, textural defects, and reduced consistency in baked products. The absence of integrated temperature monitoring further forces users to rely on manual checks, hindering the ability to experiment reliably with specific time-temperature fermentation profiles.

Several deficiencies exist in current household fermentation practices. Temperature inconsistency is common due to the absence of automated heating control. Traditional setups lack real-time temperature feedback, compelling users to perform manual measurements. Many improvised warming methods are energy-inefficient, and loosely covered or exposed containers may increase contamination risks from airborne microorganisms or insects. Existing household fermentation solutions are also typically tailored to solid or semi-solid dough and are not well suited to liquid dough, which requires secure containment and uniform temperature distribution to avoid thermal stratification.

Additional limitations of known household solutions include significant space requirements for water baths or heating arrangements, difficulty maintaining stable temperatures over extended fermentation periods, and the need for substantial user expertise. These limitations discourage novice users and reduce the reliability of fermentation results.

The increasing consumer interest in artisanal baking and home food preparation has created demand for compact, accessible devices capable of providing the precision and consistency of commercial fermentation equipment in a household setting. Consumers seek tools that enable the preparation of fermented products with improved temperature stability, repeatability, and control while remaining affordable and easy to operate.

Existing household fermentation devices, however, commonly lack adequate insulation, automated temperature control, or integrated temperature sensing. They often provide insufficient thermal retention, resulting in continuous heat loss and sensitivity to ambient temperature fluctuations. Many lack user-programmable parameters, digital interfaces, or feedback-based temperature adjustment mechanisms. These limitations are particularly problematic for liquid dough fermentation, which requires accurate internal temperature measurement, secure containment, and even thermal distribution across the entire fluid mass.

### DESCRIPTION OF THE RELATED ART

U.S. Patent Application Publication No. US 2023/0323260, Junhui Li describes a fermentation device including a fermentation tank with an insulating jacket, a heating element, and a temperature-control system. The insulating structure of the reference, however, does not disclose a multilayer insulation assembly of the type describtoed herein, nor does it describe the specific insulation materials or layer configuration disclosed in the present application. The referenced device is further directed to beer fermentation, and the arrangement of its components is adapted for that purpose.

U.S. Pat. No. 9,523,067 and U.S. Patent Application Publication No. US 2012/0189736 disclose temperature-controlled fermenting containers comprising a container, a controller, a temperature sensor, a heating mechanism, and an insulated portion. These references also describe timing mechanisms for regulating fermentation parameters. The disclosed containers, however, are not described as stainless-steel containers, nor do the references disclose a multilayer insulated waterproof bag or a fastening mechanism such as a hook-and-loop (Velcro) closure. The references further do not describe a configuration specifically adapted for liquid dough fermentation.

U.S. Patent Application Publication No. US 2005/0144696 relates to an adjustably insulative construct comprising multiple layers including cotton fabric and, in some embodiments, polyurethane film. While the reference describes layered insulation materials, it does not disclose the use of such constructs in connection with fermentation devices, nor does it describe an insulated structure configured to house a fermentation container.

U.S. Patent Application Publication No. US 2004/0040087, Craig Lack et al. disclose a multilayer insulating waterproof material that includes, among other layers, cotton insulation and a waterproof layer. Although the layered composition bears similarity to certain aspects of the insulation materials described herein, the reference does not disclose an insulating assembly configured for use with a fermentation device, nor does it disclose a five-layer structure as described in the present application.

None of the cited prior-art references discloses a compact fermentation device specifically configured for liquid dough and incorporating a stainless-steel container within a multilayer insulated waterproof bag having integrated heating and sensor-based temperature control. The prior art further does not teach a closed-loop system in which a temperature probe monitors internal dough temperature and automatically regulates heating to maintain stable fermentation conditions.

### SUMMARY OF THE INVENTION

In accordance with the present invention, there is provided a fermentation device configured for fermenting liquid dough. In some embodiments, the device comprises a fermentation container including a basin and a lid, wherein the lid couples to the basin to define an interior volume and an exterior surface. The interior volume may be configured to hold liquid dough or other fermentable substrates.

In certain embodiments, an insulation bag surrounds the exterior surface of the fermentation container. The insulation bag may include a body portion and a lid portion that together define an interior cavity. The body portion may receive the basin of the fermentation container, and the lid portion may cover the lid of the fermentation container when the device is in use.

In some embodiments, the insulation bag comprises a multilayer structure including five distinct layers. The multilayer structure may include: a first layer comprising a lining fabric positioned innermost relative to the fermentation container; a second layer comprising a heated mat; a third layer comprising an insulation film; a fourth layer comprising a cotton insulation material; and a fifth, outermost layer comprising a waterproof material. The multilayer configuration may be arranged to provide improved thermal retention, uniform heat distribution, and moisture resistance.

In various embodiments, a controller is operatively connected to the heated mat and configured to regulate the temperature of the heated mat. The controller may include a user interface configured to receive user input and provide output displays, and may further include a timer for managing timed fermentation processes.

In some embodiments, a temperature sensor is operatively coupled to the controller. The temperature sensor may include a probe that extends into the interior volume of the fermentation container and is configured to continuously measure an internal temperature of the fermenting material. The temperature sensor may generate electrical signals indicative of the internal temperature and communicate such signals to the controller.

In certain embodiments, the controller is configured to display the internal temperature on the user interface and automatically adjust the temperature of the heated mat to maintain selected fermentation conditions based on real-time temperature data received from the temperature sensor. The closed-loop feedback arrangement may support stable and consistent fermentation results.

In various embodiments, the fermentation container comprises stainless steel. The insulation bag may include a pocket configured to receive or support the controller. The lining fabric of the first layer may function as a heat-dampening material to reduce heat dispersion. The waterproof material of the fifth layer may be substantially water-impermeable. A power cord may be operatively connected to the controller and configured to provide electrical power to the controller and the heated mat.

### BRIEF DESCRIPTION OF THE DRAWING

The accompanying drawings illustrate several embodiments of the present invention and, together with the following description, serve to explain the principles of the invention according to the embodiments. One skilled in the art will recognize that the particular embodiments illustrated in the drawings are merely exemplary and are not intended to limit the scope of the present invention.

FIG. 1 is a perspective view of a fermentation device in accordance with various embodiments of the present invention, illustrating a fermentation container positioned within an insulation bag.

FIG. 2 is a perspective view of the fermentation device in its assembled configuration, showing the insulation bag and the controller.

FIG. 3 is a cross-sectional diagram illustrating the multilayer structure of the insulation bag, showing the five distinct layers from innermost to outermost.

### DETAILED DESCRIPTION OF THE INVENTION

Unless otherwise defined, all technical terms used herein relating to fermentation systems, heating elements, thermal insulation structures, temperature-control circuits, sensor assemblies, and household appliance design have the same meaning as commonly understood by one of ordinary skill in the relevant fields, including food science, thermal engineering, electrical control systems, and consumer kitchen devices. It will be further understood that terms such as “fermentation container,” “temperature sensor,” “insulation layer,” “heated mat,” “controller,” and other technical terms used throughout this specification should be interpreted in a manner consistent with their usage in the context of the described embodiments and the current state of household fermentation and thermal-control technology. These terms should not be interpreted in an idealized or overly formal sense unless expressly stated herein. For clarity and conciseness, well-known elements, functions, or constructions associated with insulated enclosures, sensor-based temperature control, or consumer appliance wiring may not be described in exhaustive detail.

The terminology used herein is intended to describe particular embodiments of the fermentation device and is not intended to be limiting. As used herein, the singular forms “a layer,” “a controller,” “a fermentation container,” “a probe,” or “a heated mat” are intended to include plural forms unless the context clearly dictates otherwise. Similarly, references to materials such as “insulation film,” “cotton material,” or “waterproof material” should be understood to encompass one or multiple sections, segments, or variations of such materials. Descriptions of temperature measurements, heating operations, or sensor readings should be understood to include repeated or continuous occurrences unless otherwise specified.

With reference to the use of the terms “comprise,” “comprises,” or “comprising” when describing the components, assemblies, or features of the fermentation device and in the following claims, these terms are intended to be interpreted inclusively rather than exclusively. For example, a description stating that the insulation bag “comprises five layers” should be understood to include the recited layers but not to exclude additional intermediate, adjacent, or supplemental layers unless explicitly stated. Likewise, a description stating that the device “comprises a controller” should be understood to permit the use of additional control elements, sensors, or interfaces not expressly mentioned in the description.

Furthermore, terms such as “connected,” “coupled,” “secured,” “enclosed,” “supported,” or “in communication with,” when describing the interaction between the heated mat, controller, sensor probe, or insulation layers, should be interpreted to include both direct and indirect connections or relationships through one or more intermediary elements, unless expressly stated otherwise. References to operations such as “measuring,” “regulating,” “heating,” or “maintaining” should be understood to encompass both continuous and intermittent processes, as well as feedback-controlled or manually adjusted implementations, unless specifically limited within the context.

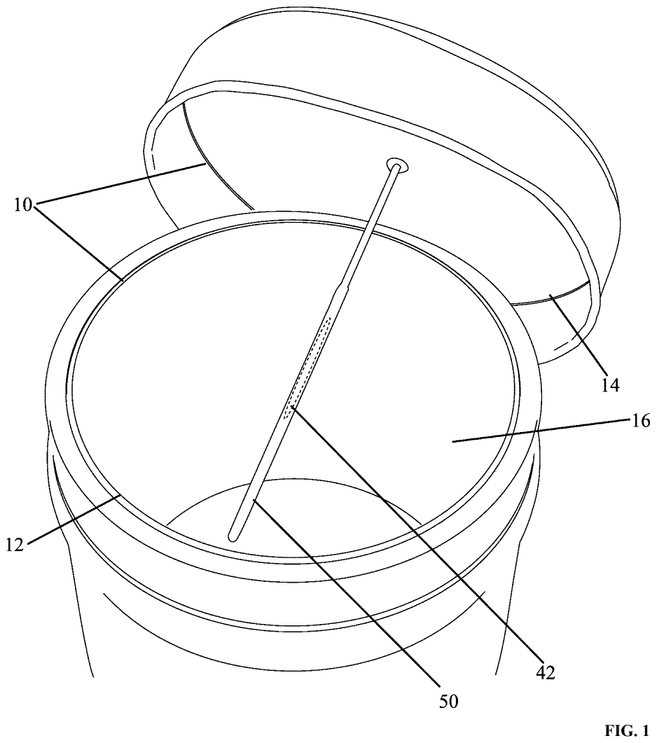

In various embodiments, and as illustrated in FIG. 1, the fermentation device comprises a fermentation container 10 configured to hold a fermentable substrate. The fermentation container 10 includes a basin 12 and a lid 14. The lid 14 is configured to couple to the basin 12 so as to define an interior volume 16. The interior volume 16 is adapted to contain liquid dough or other fermentable materials.

The basin 12 may be provided in a variety of shapes, sizes, and capacities depending on the intended application. For household use, the basin 12 typically has a capacity of approximately 0.5 liters to 5 liters, and more preferably between 1 liter and 3 liters. The basin 12 may be formed with a cylindrical profile, a rectangular profile, or any other suitable geometric configuration. In a preferred embodiment, the basin 12 is cylindrical and includes a wide-mouth opening configured to facilitate convenient filling and discharge of the liquid dough or other fermentable substrate.

In some embodiments, the lid 14 is removably attachable to the basin 12 to permit user access. The lid 14 may include a sealing element such as a gasket to reduce contamination or moisture loss. In certain embodiments, the lid 14 includes an opening or grommet through which a temperature sensor probe 50 is inserted into the interior volume 16. In alternative embodiments the lid 14 may include a pressure release valve to allow gases produced during fermentation to escape while preventing external air from entering.

In preferred embodiments, the fermentation container 10 comprises stainless steel, such as Type 304 or Type 316. In other embodiments, the fermentation container 10 may comprise glass, ceramic, or food-grade polymers

As shown in FIG. 1, the temperature sensor probe 50 extends into the interior volume 16 and is operatively coupled to a temperature sensor 42. The probe 50 is positioned to sense the internal temperature of the fermenting substrate rather than ambient air or surface temperatures

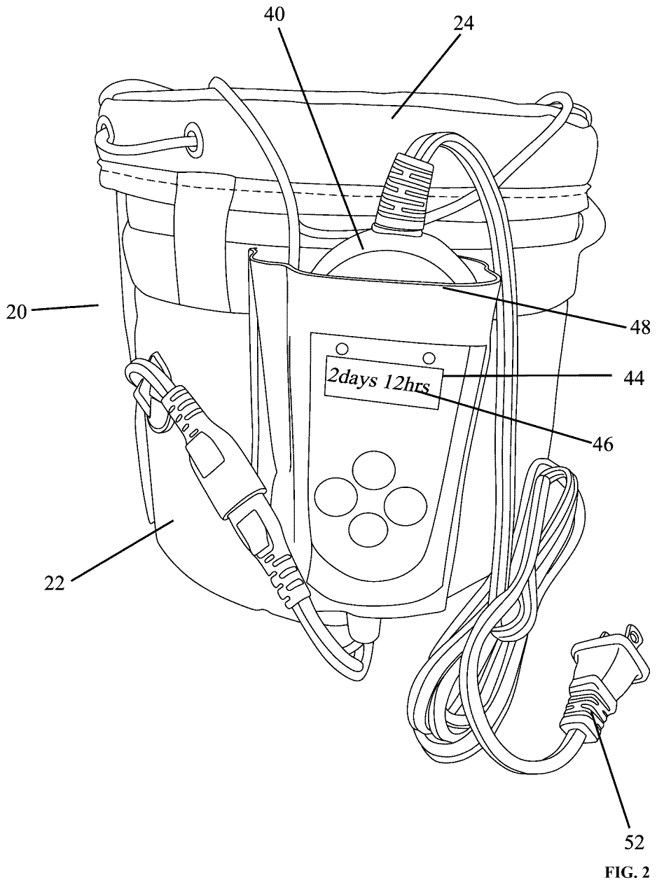

As illustrated in FIG. 2, the fermentation container 10 is positioned within an insulation bag 20. The insulation bag 20 includes a body portion 22 and a lid portion 24, which together define an interior cavity sized to receive the fermentation container 10.

In some embodiments, the body portion 22 and lid portion 24 are configured to open and close using a fastening mechanism such as a zipper, hook-and-loop fastener, magnetic closure, or snap system. The body portion 22 may surround the basin 12, and the lid portion 24 may cover the lid 14, thereby enclosing the fermentation container 10 within an insulated environment.

A power cord 52 may be connected to the controller 40 to supply electrical power to both the controller 40 and a heating element located within the insulation bag 20. The device may operate with household AC power or other suitable power sources depending on the embodiment.

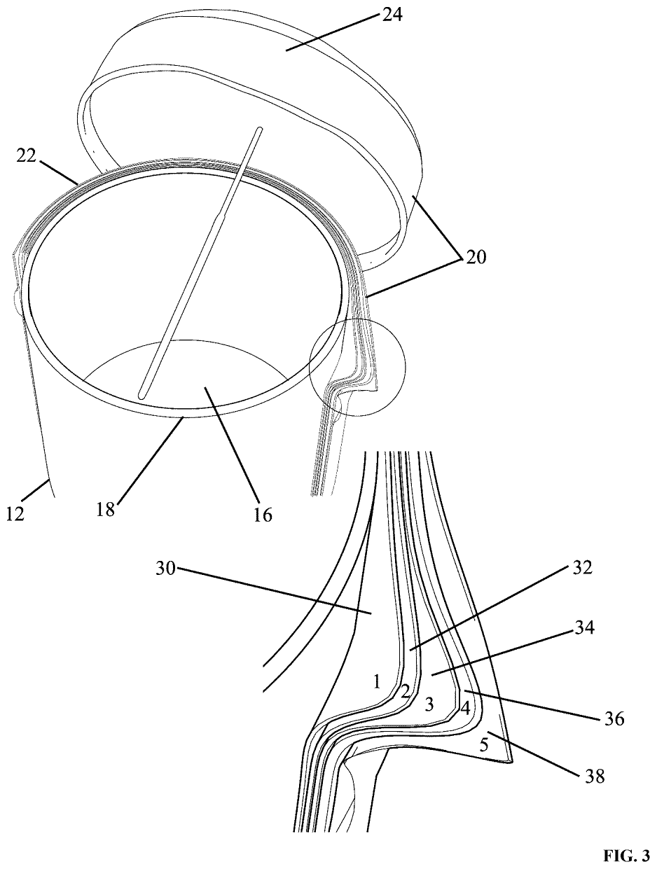

As shown in FIG. 3, the insulation bag 20 comprises, in various embodiments, a multilayer structure including five distinct layers arranged from innermost to outermost.

In some embodiments, the first layer 30 comprises a lining fabric positioned adjacent to the exterior surface 18 of the fermentation container 10. The lining fabric is selected for its heat-dampening characteristics, thereby reducing rapid thermal dissipation from the heated mat 32 toward the exterior. In preferred embodiments, the lining fabric comprises cotton flannel, cotton fleece, synthetic fleece, or other materials exhibiting comparable thermal-buffering properties. The first layer 30 provides a soft protective interface that prevents direct contact between the fermentation container 10 and the heated mat 32 while contributing initial thermal insulation.

In certain embodiments, the second layer comprises the heated mat 32, which constitutes the active heating element of the device. The heated mat 32 is a flexible, electrically resistive heating structure configured to generate controlled heat upon application of electrical current. The heated mat 32 may employ various heating technologies, including but not limited to resistive wire elements embedded within a flexible silicone or polymer substrate, carbon-fiber heating elements, conductive textile heating elements, or printed-circuit heating elements. The heated mat 32 is dimensioned and contoured to correspond to the profile of the fermentation container 10 so as to promote uniform heat distribution. In preferred embodiments, the heated mat 32 substantially surrounds the sidewall and bottom region of the basin 12 of the fermentation container 10.

In various embodiments, the third layer comprises an insulation film 34 configured to serve as a thermal barrier that minimizes heat loss from the heated mat 32 to the surrounding environment. The insulation film 34 may comprise closed-cell foam materials such as ethylene-vinyl acetate (EVA) foam, polyethylene foam, or neoprene; metallized reflective films or aerogel-based insulation. The insulation film 34 enhances the thermal efficiency of the device by reducing the energy required to achieve and maintain a desired fermentation temperature.

In some embodiments, the fourth layer comprises a cotton material 36 that provides supplemental thermal insulation through the natural air-retentive properties of cotton fibers. The cotton material 36 also imparts structural support and shape stability to the insulation bag 20. Suitable cotton materials include cotton batting, cotton wadding, quilted cotton fabrics, or dense cotton canvas. Additionally, the fourth layer 36 provides cushioning to protect the fermentation container 10 during handling and transport.

In certain embodiments, the fifth layer comprises a waterproof material 38 disposed as the outermost layer of the insulation bag 20. The waterproof material 38 is substantially impermeable to moisture, thereby preventing ingress of water into the internal layers and protecting the electrical components such as the heated mat 32, controller, and associated sensors from moisture-related damage. Suitable materials for the waterproof layer include waterproof nylon fabrics, polyurethane-coated textiles, polyvinyl chloride (PVC) fabrics, or laminated waterproof composites. The waterproof material 38 further provides a durable, cleanable exterior surface resistant to wear, staining, and environmental exposure.

In various embodiments the fermentation device includes a temperature control system configured to maintain selected thermal conditions within the interior volume 16 of the fermentation container 10. The temperature control system may generally comprise a controller 40, a temperature sensor 42, and the heated mat 32 positioned within the multilayer insulation structure of the insulation bag 20.

In some embodiments, the controller 40 is operatively coupled to the heated mat 32 and is configured to regulate heat output by modulating electrical power supplied to the heated mat 32. The controller 40 may include electronic circuitry such as a microprocessor or microcontroller, power regulation components e.g., a triac, silicon-controlled rectifier (SCR), or pulse-width modulation circuitry, and interface circuitry enabling user interaction.

As illustrated in FIG. 2, the controller 40 may include a user interface 44 configured to receive user input and generate visual output. In certain embodiments, the user interface 44 comprises a digital display for presenting real-time information such as internal temperature, target temperature settings, elapsed fermentation time, and remaining time. Input elements such as buttons, touch-sensitive areas, or a touchscreen may enable the user to select a desired fermentation temperature, for example within the range of approximately 20° C. to 45° C., and to set a fermentation duration, for example between 1 hour and 48 hours.

In some embodiments, the controller 40 further includes a timer configured to support timed fermentation cycles. The timer may count down a selected duration and provide a visual or audible notification when the fermentation period has concluded.

As shown in FIG. 2, the controller 40 may be positioned within a controller pocket 48 provided on or incorporated into the insulation bag 20. The pocket 48 may be positioned on an exterior surface of the insulation bag 20 for convenient user access, or alternatively within the interior cavity of the insulation bag 20 to reduce exposure to ambient environmental conditions. The pocket 48 may retain the controller 40 securely during operation or transport.

In various embodiments, the temperature sensor 42 is operatively connected to the controller 40 via electrical conductors. As shown in FIG. 1, the temperature sensor 42 includes a probe 50 that extends into the interior volume 16 of the fermentation container 10. The probe 50 may be positioned such that its sensing region is in direct contact with or immersed in the fermenting material, allowing measurement of the actual internal temperature of the substrate rather than ambient air.

The temperature sensor 42 may be implemented using various sensing technologies, including but not limited to thermistors, thermocouples, resistance temperature detectors (RTDs), or semiconductor temperature sensors. In certain embodiments, the temperature sensor 42 comprises a waterproof, food-safe thermistor enclosed in a stainless-steel probe housing. The sensor 42 may continuously or periodically measure the internal temperature at predetermined intervals and generate corresponding electrical signals for communication to the controller 40.

In some embodiments, a power cord 52 is operatively connected to the controller 40 and configured to supply electrical power to both the controller 40 and the heated mat 32. The power cord 52 may terminate in a plug compatible with household electrical outlets. In embodiments intended for use in the United States, the plug may be configured for approximately 120V AC power; for international applications, the plug may be configured for 220-240V AC systems. The controller 40 may additionally incorporate circuitry for converting AC input power to appropriate DC operating voltages for internal electronic components.

As shown in FIGS. 1-3, the controller 40 and temperature sensor 42 may operate cooperatively in a closed-loop temperature regulation system. The controller 40 may be configured to display the internal temperature measured by the temperature sensor 42 on the user interface 44, thereby providing real-time feedback to the user.

In operation, the controller 40 may continuously compare the measured internal temperature within the interior volume 16 to a user-selected target temperature. If the measured temperature falls below the target temperature, the controller 40 may increase electrical power supplied to the heated mat 32 to raise the temperature. If the measured temperature exceeds the target temperature, the controller 40 may reduce or interrupt electrical power to the heated mat 32 to allow cooling.

In some embodiments, the controller 40 may employ proportional-integral-derivative (PID) control, on-off control logic with hysteresis, or other temperature regulation algorithms to maintain the internal temperature within a predetermined range. The use of this automated closed-loop control system may maintain fermentation conditions within approximately ±0.5° C. to ±2° C. of the target temperature, thereby providing stable and consistent thermal conditions during the fermentation process.

In alternative embodiments, the materials of the multilayer insulation structure may be varied while maintaining the functional arrangement of multiple distinct insulating layers. Suitable alternatives include wool batting, synthetic insulation materials such as Thinsulate™, vacuum insulation panels, metallized reflective films, or combinations thereof.

In certain embodiments, the heated mat may employ alternative heating technologies, including flexible silicone heaters, Peltier thermoelectric modules, carbon-fiber heating films, induction-based heating elements, or printed resistive heating circuits.

In various embodiments, the controller may include additional features such as wireless connectivity (e.g., Wi-Fi, Bluetooth), remote monitoring via mobile applications, preprogrammed fermentation profiles, data logging, or integration with smart home systems.

In some embodiments, the device may include multiple fermentation containers positioned within a single insulation bag to enable simultaneous fermentation of different materials at the same or independent temperature settings.

Alternative embodiments may incorporate a gentle agitation, vibration, or stirring mechanism configured to periodically mix the fermenting material to enhance uniformity of temperature distribution and prevent particle settling.

In some embodiments, operation may begin with preparation of a fermentable substrate. The user may prepare liquid dough or other fermentable material according to a desired formulation. Exemplary substrates include, without limitation, pancake batter, waffle batter, crêpe batter, sourdough starter, idli batter, dosa batter, and other flowable or semi-flowable mixtures suitable for fermentation.

The user may then open the lid 14 of the fermentation container 10 and introduce the liquid dough into the basin 12. The liquid level may be selected to allow adequate space for volumetric expansion during fermentation, such as between approximately 50% and 80% of the basin capacity in certain embodiments.

In some embodiments, the probe 50 of the temperature sensor 42 may be inserted through an opening in the lid 14 such that the probe is at least partially immersed within the liquid dough. This arrangement may facilitate accurate monitoring of the internal temperature of the fermenting material.

After the liquid dough is introduced, the lid 14 may be closed and secured to define the interior volume 16 of the fermentation container 10. The fermentation container 10 may then be placed within the interior cavity of the insulation bag 20, and the lid portion 24 of the insulation bag 20 may be closed using a fastening mechanism such as a zipper, hook-and-loop fastener, or other closure structure.

In certain embodiments, the user may connect the power cord 52 to an electrical outlet to supply power to the controller 40 and heated mat 32. The controller 40, via the user interface 44, may allow the user to select a target fermentation temperature and a fermentation duration. Typical fermentation temperatures may range from approximately 24° C. to 32° C., although higher or lower temperatures may be selected depending on the type of fermentable substrate and the desired characteristics.

Upon user activation through the user interface 44, the controller 40 may initiate operation of the heated mat 32. During operation, the controller 40 may receive continuous or periodic temperature data from the temperature sensor and may automatically adjust the heating output to maintain the target temperature. The user interface 44 may display real-time temperature readings, elapsed time, and other operational parameters.

In some embodiments, upon expiration of the user-selected fermentation duration, the controller 40 may activate a visual or audible alert to indicate completion. The user may then open the insulation bag 20, remove the fermentation container 10, and access the fermented substrate by opening the lid 14.

After fermentation is complete, the fermentation container 10 may be cleaned using conventional dishwashing techniques. The exterior of the insulation bag 20 may be cleaned using a damp cloth or similar cleaning method, while avoiding exposure of the controller 40 and related electronic components to liquid.

The present invention provides controlled thermal management through automated temperature regulation and an internal sensor-based feedback loop, enabling stable and reproducible fermentation conditions. The multilayer insulation structure minimizes heat loss and reduces overall energy consumption, while the sealed container and enclosed construction help limit exposure to airborne contaminants and environmental fluctuations.

The device further supports accessible and efficient operation. The user interface allows for simple parameter selection, and automated regulation reduces the need for user intervention throughout the fermentation cycle. The compact physical configuration is suitable for household environments and can be stored easily when not in use. Structural and electronic features, including integrated heating control and a waterproof exterior layer, may reduce risks associated with overheating or moisture exposure.

In addition, the device accommodates a wide range of fermentable liquid dough formulations and may facilitate consistent preparation across varying ambient conditions. By simplifying the fermentation process and providing predictable results, the invention supports the preparation of fermented foods that may retain desirable textural, sensory, and nutritional characteristics.

The fermentation device described herein provides an improved system for maintaining controlled fermentation conditions through the combined use of a dedicated fermentation container, a multilayer insulation structure, and an automated temperature regulation system. The embodiments presented illustrate how the device may be implemented to support consistent and reliable fermentation in a household environment while accommodating variations in design, materials, and operational features.

The foregoing description is intended to illustrate, rather than limit, the various embodiments of the invention. It will be understood by those skilled in the art that modifications, substitutions, and variations may be made without departing from the scope of the invention as defined by the appended claims. Structural and functional features described in connection with one embodiment may be incorporated into other embodiments unless stated otherwise, and all such variations are considered to fall within the scope of the present disclosure.

It is therefore to be understood that the present invention encompasses all embodiments and equivalents falling within the spirit and scope of the claims that follow.

What is claimed is:

1. A fermentation device for fermenting liquid dough or other fermentable substrates, comprising:
a fermentation container comprising a basin and a lid, wherein the lid is coupled to the basin to define an interior volume and an exterior surface, the interior volume configured to hold liquid dough or other fermentable substrates;
an insulation bag surrounding the exterior surface of the fermentation container, the insulation bag comprising:
a body portion and a lid portion that together define an interior cavity, wherein the basin of the fermentation container is contained within the body portion of the interior cavity and the lid of the fermentation container is covered by the lid portion of the insulation bag; and
a multilayer structure comprising a plurality of layers including:
a first layer comprising a lining fabric positioned innermost relative to the fermentation container;
a second layer comprising a heated mat;
a third layer comprising an insulation film;
a fourth layer comprising a cotton material; and
a fifth layer comprising a waterproof material positioned outermost;
a controller operatively connected to the heated mat and configured to regulate a temperature of the heated mat, the controller comprising:
a user interface configured to receive user input and generate output displays; and
a timer configured to facilitate timed fermentation processes;
a temperature sensor operatively coupled to the controller, the temperature sensor comprising a probe that extends into the interior volume of the fermentation container and is configured to:
continuously measure an internal temperature of the interior volume; and
communicate electrical signals indicative of the internal temperature to the controller;
wherein the controller is configured to display the internal temperature on the user interface and automatically adjust the temperature of the heated mat to maintain selected fermentation conditions within the interior volume.

2. The fermentation device of claim 1, wherein the fermentation container comprises stainless steel.

3. The fermentation device of claim 1, wherein the insulation bag further comprises a pocket configured to contain the controller.

4. The fermentation device of claim 1, wherein the lining fabric of the first layer is configured as a heat-dampening layer to prevent heat dispersion.

5. The fermentation device of claim 1, wherein the waterproof material of the fifth layer is substantially water-impermeable.

6. The fermentation device of claim 1, further comprising a power cord operatively connected to the controller and configured to provide electrical power to the controller and the heated mat.

7. The fermentation device of claim 1, wherein the multilayer structure is configured to provide thermal retention to maintain a stable environment for fermenting liquid dough or other fermentable substrates.

8. The fermentation device of claim 1, wherein the heated mat is positioned between the lining fabric and the insulation film to provide uniform heat distribution to the fermentation container.

9. The fermentation device of claim 1, wherein the controller is configured to automatically regulate the temperature of the heated mat based on real-time temperature data received from the temperature sensor.

10. The fermentation device of claim 1, wherein the user interface comprises a display screen configured to show the internal temperature and allow user adjustment of target fermentation temperature and timing parameters.

11. A fermentation device for household use configured for fermenting liquid dough or other fermentable substrates, comprising:
a fermentation container comprising:
a basin defining an interior volume; and
a lid coupled to the basin to enclose the interior volume;
a multilayer insulation bag enclosing the fermentation container, the multilayer insulation bag comprising:
an innermost lining fabric layer;
a heated mat layer positioned adjacent to the lining fabric layer;
an insulation film layer;
a cotton material layer; and
an outermost waterproof layer;
a temperature control system comprising:
a controller having a user interface and a timer;
the heated mat operatively connected to the controller; and
a temperature sensor having a probe extending into the interior volume of the fermentation container, the temperature sensor configured to communicate temperature data to the controller;
wherein the controller is configured to maintain a predetermined fermentation temperature within the interior volume by automatically adjusting power supplied to the heated mat based on the temperature data.

12. The fermentation device of claim 11, wherein the fermentation container comprises stainless steel.

13. The fermentation device of claim 11, wherein the multilayer insulation bag comprises a body portion and a lid portion that couple together to form an interior cavity housing the fermentation container.

14. The fermentation device of claim 11, wherein the lining fabric layer functions as a heat-dampening layer to reduce heat dispersion.

15. The fermentation device of claim 11, further comprising a power cord connected to the controller for supplying electrical power.

16. The fermentation device of claim 11, wherein the insulation bag further comprises a controller pocket integrated into the bag.

17. The fermentation device of claim 11, wherein the temperature sensor continuously monitors the internal temperature and provides real-time feedback to the controller for precise temperature regulation.

18. A method of fermenting liquid dough or other fermentable substrates using a controlled fermentation device, the method comprising:
providing a fermentation device comprising:
a fermentation container with a basin and a lid defining an interior volume;
a multilayer insulation bag surrounding the fermentation container, the multilayer insulation bag including a heated mat layer;
a controller operatively connected to the heated mat; and
a temperature sensor having a probe extending into the interior volume;
placing liquid dough or another fermentable substrate into the interior volume of the fermentation container;
closing the lid of the fermentation container to enclose the substrate;
setting a target fermentation temperature and duration via a user interface of the controller;
activating the controller to initiate a fermentation process;
continuously monitoring an internal temperature of the interior volume via the temperature sensor;
automatically regulating power to the heated mat based on temperature data from the temperature sensor to maintain the target fermentation temperature;
displaying the internal temperature on the user interface during fermentation; and
terminating the fermentation process upon expiration of the fermentation duration.

19. The method of claim 18, wherein the multilayer insulation bag provides thermal retention to maintain stable fermentation conditions and reduce energy consumption.

20. The method of claim 18, wherein automatically regulating power to the heated mat comprises increasing power when the internal temperature falls below the target fermentation temperature and decreasing power when the internal temperature exceeds the target fermentation temperature.
